(19) United States
(12) Patent Application Publication
(10) Pub. No.: US 2021/0252013 A1
(43) Pub. Date: Aug. 19, 2021

(54) METHODS OF TREATING CANCER WITH PI3K INHIBITOR, GDC-0077

(71) Applicant: Genentech, Inc., South San Francisco, CA (US)

(72) Inventors: Susan Greene, South San Francisco, CA (US); Stephanie Joo, South San Francisco, CA (US); Jennifer Schutzman, South San Francisco, CA (US)

(73) Assignee: Genentech, Inc., South San Francisco, CA (US)

(21) Appl. No.: 17/156,381

(22) Filed: Jan. 22, 2021

Related U.S. Application Data
(63) Continuation of application No. PCT/US2019/042539, filed on Jul. 19, 2019.
(60) Provisional application No. 62/742,636, filed on Oct. 8, 2018.
(60) Provisional application No. 62/702,197, filed on Jul. 23, 2018.

(51) Int. Cl. A61K 31/553; A61K 45/06; A61P 35/00; A61K 31/519; A61K 31/565

(52) U.S. Cl. CPC A61K 31/553; A61K 45/06; A61K 31/565; A61K 31/519; A61P 35/00

(57) ABSTRACT

Described herein are methods of treating PIK3CA-mutant cancer patients by administering metformin and a PI3K inhibitor, GDC-0077.

### CROSS-REFERENCE TO RELATED APPLICATIONS

This application claims priority benefit of U.S. Provisional Applications, Ser. No. 62/702,197 filed on 23 Jul. 2018 and Ser. No. 62/742,636 filed on 8 Oct. 2018, which are incorporated herein in their entirety.

### FIELD OF THE INVENTION

The invention relates generally to treatment of PIK3CA-mutant cancer patients by administering metformin and a PI3K inhibitor, GDC-0077.

### BACKGROUND OF THE INVENTION

Phosphatidylinositol 3-kinase (PI3K) is a lipid kinase that upon activation by growth factor receptors and integrins regulates cell proliferation, survival, and migration. PI3K catalyzes the phosphorylation of phosphatidylinositol-4,5-bisphosphate (PIP₂) to generate phosphatidylinositol-3,4,5-triphosphate (PIP₃), a second messenger involved in the phosphorylation of AKT and other components in the AKT/mTOR pathway (Cantley L C Science (2002) 296(5573):1655-1657; Guertin D A, et al (2007) Cancer Cell 12:9-22). PI3K and its downstream effectors, AKT and mTOR, are major nodes in the PI3K/AKT/mTOR signaling pathway and are critical for cell-cycle modulation, cell growth, metabolism, motility, and survival (Rameh, et al (1999) J Biol Chem. 274:8347-8350; Cantrell D A (2001) J Cell Sci 114:1439-1445; Hanahan D, et al (2011) Cell 144:646-674; Vanhaesebroeck B, et al (2012) Nat Rev Mol Cell Biol 13:195-203).

PI3K is a heterodimer consisting of p85 and p110 subunits (Otsu et al (1991) Cell 65:91-104; Hiles et al (1992) Cell 70:419-429). Four distinct Class I PI3Ks have been identified, designated PI3K α (alpha), β (beta), δ (delta), and γ (gamma), each consisting of a distinct 110 kDa catalytic subunit and a regulatory subunit p85. These four isoforms are the product of four genes: PIK3CA, PIK3CB, PIK3CD, and PIK3CG. Three of the catalytic subunits, i.e., p110 alpha, p110 beta and p110 delta, each interact with the same regulatory subunit, p85; whereas p110 gamma interacts with a distinct regulatory subunit, p101. The patterns of expression of each of these PI3Ks in human cells and tissues are distinct. In each of the PI3K alpha, beta, and delta subtypes, the p85 subunit acts to localize PI3K to the plasma membrane by the interaction of its SH2 domain with phosphorylated tyrosine residues (present in an appropriate sequence context) in target proteins (Rameh et al (1995) Cell, 83:821-30; Volinia et al (1992) Oncogene, 7:789-93).

Dysregulation of the PI3K/AKT/mTOR signaling pathway through multiple different mechanisms has been described in solid tumor malignancies, including activating and transforming mutations, as well as amplification, of PIK3CA that encodes the p110 alpha subunit of PI3K (Gustin J, P et al (2008) Curr Cancer Drug Targets 8:733-740; Yuan T L, (2008) Oncogene 27:5497-5510, Courtney K D, et al (2010) J Clin Oncol 28:1075-1083). Activating mutations in the PIK3CA gene occur primarily in exons 9 and 20 (“hotspot” regions), which encode the helical and kinase domains of PI3K alpha protein (Bachman K E, et al (2004) Cancer Biol Ther 3:772-5; Samuels Y, et al (2004) Science 304:554).

Up to 70% of breast cancers have some form of molecular aberration of the PI3K/AKT/mTOR pathway (Cancer Genome Atlas Network 2012). Hyperactivation of the PI3K/AKT/mTOR signaling pathway was shown to promote both de novo and acquired resistance to endocrine therapy in ER+ breast cancer cell lines and xenograft models (Sabnis G, et al (2007) Clin Cancer Res 13:2751-2757), and simultaneous blocking of the PI3K/AKT/mTOR pathway enhances anti-tumor activity (Boulay A, et al (2005) Clin Cancer Res 11:5319-5328), indicating blocking PI3K/AKT/mTOR pathway signaling may have a therapeutic benefit in patients with ER+ breast cancer.

The PI3K/AKT/PTEN pathway is an attractive target for cancer drug development since such agents would be expected to inhibit cellular proliferation, to repress signals from stromal cells that provide for survival and chemoresistance of cancer cells, to reverse the repression of apoptosis and surmount intrinsic resistance of cancer cells to cytotoxic agents. There is a need for additional modulators of PI3Kα (alpha isoform) that are useful for treating cancers, particularly an inhibitor of PI3Kα that is selective for mutant PI3Kα expressing tumors relative to non-mutant PI3Kα expressing cells. There is especially a need for such an agent that selectively inhibits the PI3Kα isoform relative to the PI3Kβ, PI3Kδ, and PI3Kγ isoforms, which may be expected to result in an enhanced therapeutic window.

Hyperglycemia is a dose-limiting toxicity associated with treatment with PI3K alpha inhibitors (Juric D, et al (2013) Proceedings of the 104th Annual Meeting of the American Association for Cancer Research; 2013 Apr. 6-10; Washington, D.C. Philadelphia (Pa.): AACR; Cancer Res 2013b; 73(8 Suppl): Abstract nr LB-64). Management guidelines for hyperglycemia with PI3K pathway inhibitors have recommended metformin as the first-line treatment (Hostalek U, et al (2015) Drugs 75:1071-1094; Busaidy et al (2012) J Clin Oncol 30:2919-28). Mitigation or management of a hyperglycemic effect may provide additional opportunities for treatment of cancer with PI3K alpha inhibitors. Maximizing therapeutic benefit, while minimizing treatment-related toxicities, is particularly important in HR+/HER2 negative breast cancer where treatment times can be long.

### SUMMARY OF THE INVENTION

The invention provides methods of treating patients with cancer with a PI3K inhibitor, GDC-0077, after treatment with the anti-hyperglycemic medication metformin to mitigate or manage hyperglycemia.

An aspect of the invention is a method for the treatment of cancer in a patient comprising administering a therapeutically effective amount of GDC-0077, or a pharmaceutically acceptable salt thereof, wherein the patient has been previously treated with metformin, and GDC-0077 has the structure:

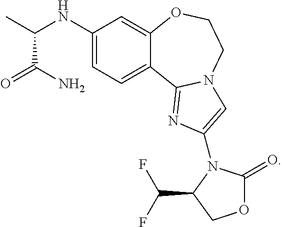 [SMILES: C[C@H](Nc1ccc2c(c1)OCCn1cc(N3C(=O)OC[C@H]3C(F)F)nc1-2)C(N)=O]

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### Definitions

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “detection” includes any means of detecting, including direct and indirect detection.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

“Patient response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Change” or “modulation” of the status of a biomarker, including a PIK3CA mutation or set of PIK3CA mutations, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry (IHC), immunocytochemistry, ELISA, or mass spectrometry whereby degradation, stabilization, or post-translational modifications of the proteins such as phosphorylation or ubiquitination is detected.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

A “chemotherapeutic agent” is a biological (large molecule) or chemical (small molecule) compound useful in the treatment of cancer, regardless of mechanism of action.

The term “mammal” includes, but is not limited to, humans, mice, rats, guinea pigs, monkeys, dogs, cats, horses, cows, pigs and sheep.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

The term “synergistic” as used herein refers to a therapeutic combination which is more effective than the additive effects of the two or more single agents. A determination of a synergistic interaction between a compound of GDC-0077 or a pharmaceutically acceptable salt thereof, and one or more chemotherapeutic agent may be based on the results obtained from the assays described herein. The results of these assays can be analyzed using the Chou and Talalay combination method and Dose-Effect Analysis with CalcuSyn® software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehir et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations.

### Clinical Trial Drugs

There are five investigational medicinal products (IMP) to be used in this trial: GDC-0077, palbociclib (IBRANCE®, Pfizer Co.), letrozole (FEMARA, Novartis), fulvestrant (FASLODEX®, AstraZeneca), and metformin.

### GDC-0077:

GDC-0077 is a potent, orally bioavailable, clinical-stage, selective inhibitor of the Class I PI3K alpha isoform, with >300-fold less potent biochemical inhibition for other Class I PI3K beta, delta, and gamma isoforms and increased potency in tumor cells bearing mutant PI3K over wild type (WT) PI3K cells (Braun, M. et al “Discovery of GDC-0077: A highly selective inhibitor of PI3K-alpha that induces degradation of mutant-p110 alpha protein” Abstracts of Papers, 254th ACS National Meeting& Exposition, Washington, D.C., USA, Aug. 20-24, 2017, MEDI-22; Garland, K. et al “Discovery of novel class of alpha selective PI3K inhibitors” Abstracts of Papers, 254th ACS National Meeting & Exposition, Washington, D.C., USA, Aug. 20-24, 2017, MEDI-103; Hong, R. et al “GDC-0077 is a selective PI3K alpha inhibitor that demonstrates robust efficacy in PIK3CA mutant breast cancer models as a single agent and in combination with standard of care therapies” 2017 San Antonio Breast Cancer Symposium, Dec. 5-9 2017, San Antonio, Tex., Abstract Publication Number: PD4-14; Edgar, K. et al “Preclinical characterization of GDC-0077, a specific PI3K alpha inhibitor in early clinical development” Cancer Research 77(13 Supplement): Abstract 156 Jul. 2017).

GDC-0077 (CAS Registry Number 2060571-02-8, Genentech, Inc., U.S. Pat. No. 9,650,393; named as (S)-2-((2-((S)-4-(difluoromethyl)-2-oxooxazolidin-3-yl)-5,6-dihydrobenzo[f]imidazo[1,2-d][1,4]oxazepin-9-yl)amino)propanamide, has the structure:

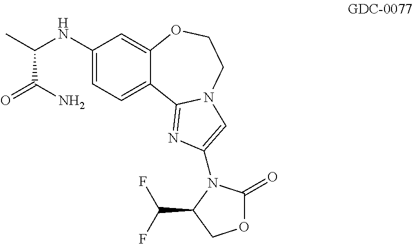 [SMILES: C[C@H](Nc1ccc2c(c1)OCCn1cc(N3C(=O)OC[C@H]3C(F)F)nc1-2)C(N)=O]

GDC-0077 exerts its activity by binding to the ATP binding site of PI3K, thereby inhibiting the phosphorylation of membrane-bound 4,5-phosphatidylinositol bisphosphate (PIP₂) to 3,4,5-phosphatidylinositol triphosphate (PIP₃). Inhibiting the phosphorylation of PIP₂ to PIP₃ decreases downstream activation of AKT and pS6, resulting in decreased cellular proliferation, metabolism, and angiogenesis. Nonclinical studies demonstrate that GDC-0077 specifically degrades mutant p110 alpha, inhibits proliferation and induces apoptosis of PIK3CA-mutant breast cancer cell lines, inhibits tumor growth in human breast xenograft models harboring PIK3CA mutations, and reduces downstream PI3K-pathway markers, including pAKT (phosphorylated form of AKT), pPRAS40, and pS6.

### Fulvestrant:

Fulvestrant is an ER antagonist and an effective treatment for postmenopausal patients with HR+ breast cancer that is relatively well tolerated. The expected toxicities for GDC-0077 and fulvestrant are not overlapping. It is important to test GDC-0077 in combination with both letrozole and fulvestrant, as these endocrine therapies have different mechanisms of action, different PK properties, and different potential for drug-drug interactions (DDIs) with GDC-0077.

Fulvestrant (FASLODEX®, AstraZeneca, CAS Reg. No. 129453-61-8) is approved by the FDA for treatment of hormone receptor-positive (HR+) metastatic breast cancer in postmenopausal women with disease progression following anti-estrogen therapy (Kansra (2005) Mol Cell Endocrinol 239(1-2):27-36; Flemming et al (2009) Breast Cancer Res Treat. May; 115(2):255-68; Valachis et al (2010) Crit Rev Oncol Hematol. March; 73(3):220-7). Fulvestrant is an estrogen receptor (ER) antagonist with no agonist effects, which works both by down-regulating and by degrading the estrogen receptor (Croxtall (2011) Drugs 71(3):363-380). Fulvestrant is also a selective estrogen receptor down-regulator (SERD).

Fulvestrant is named as (7,17P)-7-{9-[(4,4,5,5,5-pentafluoropentyl)sulfinyl]nonyl}estra-1,3,5(10)-triene-3,17-diol and has the structure:

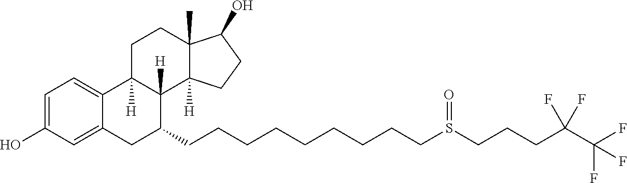

Fulvestrant belongs to a class of reversible steroidal ER antagonists that directly competes with estrogen for ER binding and is devoid of the partial agonist properties of tamoxifen. Upon binding to ER, it blocks estrogen signaling and increases the degradation of ER protein. The affinity of fulvestrant for the ER is approximately 100-fold greater than that of tamoxifen (Howell et al. (2000) Cancer 89:817-25). Fulvestrant (250 mg once monthly) was approved by the FDA in 2002 and by the EMA in 2004 for the treatment of HR-positive MBC in postmenopausal women with disease progression following anti-estrogen therapy. In multicenter Phase III studies, fulvestrant was found to be at least equivalent to anastrozole (a non-steroidal AI) in the second-line setting (Howell et al. (2002) J Clin Oncol 20:3396-3403; Osborne C K, et al (2002) J Clin Oncol 20:3386-95). Fulvestrant is also as active as tamoxifen for the first-line treatment of advanced breast cancer (Howell et al. (2004) J Clin Oncol 22:1605-1613) and displays a level of activity in patients in the post-AI metastatic disease setting similar to that of the non-steroidal AI exemestane (Chia et al. (2008) J Clin Oncol 26:1664-1670). High-dose fulvestrant (500 mg once monthly) has been demonstrated to be at least as effective as anastrozole in terms of clinical benefit rate (CBR) and overall response rate and to be associated with significantly longer time to progression for the first-line treatment of women with advanced HR-positive breast cancer (Robertson et al. (2009) J Clin Oncol 27:4530-4535). High-dose fulvestrant recently demonstrated superior progression-free survival (PFS) in women with ER-positive advanced breast cancer treated with 500 mg versus patients treated with 250 mg (Di Leo et al. (2010) J Clin Oncol 28:4594-4600). Fulvestrant (250 mg and 500 mg) was well tolerated in these studies and produced fewer estrogenic effects than did tamoxifen and resulted in less arthralgia than did the AI anastrozole (Osborne et al. (2002) J Clin Oncol 20:3386-3395). These results led to the approval of 500 mg fulvestrant given once a month as the currently approved recommended dose in the United States and the European Union (in 2010) for postmenopausal women whose disease has spread after treatment with an AI. These studies demonstrate that fulvestrant is an important treatment option for patients with advanced breast cancer and, as such, is considered appropriate control therapy for the present study.

### Palbociclib:

Palbociclib is a selective inhibitor of the cyclin-dependent kinases CDK4 and CDK6 (Finn et al (2009) Breast cancer research: BCR 11 (5):R77; Rocca et al (2014) Expert Opin Pharmacother 15 (3):407-20; U.S. Pat. Nos. 6,936,612; 7,863,278; 7,208,489; 7,456,168). Palbociclib can be prepared and characterized as described in U.S. Pat. No. 7,345,171. IBRANCE® is approved for the treatment of breast cancer.

Palbociclib (PD-0332991, IBRANCE®, Pfizer, Inc., CAS Reg. No. 571190-30-2), named as 6-acetyl-8-cyclopentyl-5-methyl-2-(5-(piperazin-1-yl)pyridin-2-ylamino)pyrido[2,3-d]pyrimidin-7(8H)-one, has the structure:

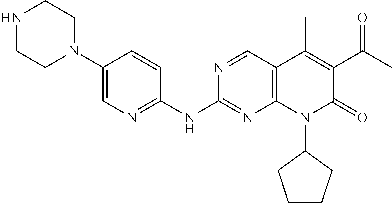 [SMILES: CC(=O)c1c(C)c2cnc(Nc3ccc(N4CCNCC4)cn3)nc2n(C2CCCC2)c1=O]

Palbociclib is a CDK4/6 inhibitor and, in combination with letrozole or fulvestrant, an effective treatment for postmenopausal patients with HR+(positive)/HER2− (negative) breast cancer. In combination with letrozole or fulvestrant, the main toxicity of palbociclib is neutropenia (Finn et al (2015) Lancet Oncol 16:25-35; Turner et al (2015) N Engl J Med 373:209-19). In combination with letrozole, 36% of patients required ≥1 dose reduction of palbociclib; dose holds and cycle delays were reported in 70% and 68% of patients, respectively (Finn et al (2016) J Clin Oncol 34(suppl; abstr 507)). In combination with fulvestrant, 34% of patients required ≥1 dose reduction of palbociclib; dose holds and cycle delays were reported in 54% and 36% of patients, respectively (Cristofanilli et al. (2016)Lancet Oncol 17:425-39). Myelosuppression is a potential toxicity of GDC-0077. In one embodiment of the study, patients enrolled in the GDC-0077 dose-escalation and dose-cohort expansion in combination with palbociclib and letrozole or in combination with palbociclib and fulvestrant have adequate screening of neutrophil, hemoglobin, and platelet counts and have CBC with differential monitored frequently throughout study treatment.

### Letrozole:

Letrozole is an effective treatment for postmenopausal patients with HR+ breast cancer that is relatively well tolerated. The expected toxicities for GDC-0077 and letrozole are not overlapping. Letrozole (FEMARA®, Novartis Pharm.) is an oral non-steroidal aromatase inhibitor for the treatment of hormonally-responsive breast cancer after surgery (Bhatnagar et al (1990) J. Steroid Biochem. and Mol. Biol. 37:1021; Lipton et al (1995) Cancer 75:2132; Goss, P. E. and Smith, R. E. (2002) Expert Rev. Anticancer Ther. 2:249-260; Lang et al (1993) The Journal of Steroid Biochem. and Mol. Biol. 44 (4-6):421-8; EP 236940; U.S. Pat. No. 4,978,672). FEMARA® is approved by the FDA for the treatment of local or metastatic breast cancer that is hormone receptor positive (HR+) or has an unknown receptor status in postmenopausal women.

Letrozole is named as 4,4′-((1H-1,2,4-triazol-1-yl)methylene)dibenzonitrile (CAS Reg. No. 112809-51-5), and has the structure:

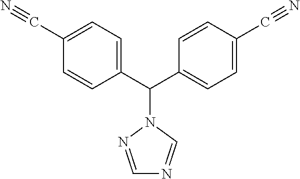 [SMILES: N#Cc1ccc(C(c2ccc(C#N)cc2)n2cncn2)cc1]

### Metformin:

Metformin, a biguanide drug, (GLUCOPHAGE®, Bristol Myers Squibb Co.) is a first-line, orally administered, prescription drug for treating type 2 diabetes in all newly diagnosed patients, unless there is evidence of renal impairment or other contraindications. (Dunning, T. et al, Diabetes Res Clin Pract. (2014) 103, 538-540). GLUCOPHAGE® (metformin hydrochloride) Tablets and GLUCOPHAGE® XR (metformin hydrochloride, Met HCl, CAS Reg. No. 1115-70-4) Extended-Release Tablets are oral anti-hyperglycemic drugs used in the management of type 2 diabetes. GLUCOVANCE® (Glyburide and Metformin HCl, Bristol Myers Squibb Co.) Tablets contain 2 oral antihyperglycemic drugs used in the management of type 2 diabetes, glyburide and metformin hydrochloride.

The anti-hyperglycemic medication metformin is an established standard-of-care treatment for Type 2 diabetes and is recommended for diabetes prevention in patients who are obese or pre-diabetic and as first-line treatment for hyperglycemia associated with PI3K pathway inhibitors (American Diabetes Association 2015; Hostalek U, et al (2015) Drugs 75:1071-1094; Busaidy N L, et al (2012) J Clin Oncol 30:2919-2928).

Metformin (pKa=12.4, CAS Reg. No. 657-24-9), also known as N,N-dimethylimidodicarbonimidic diamide and 1,1-dimethylbiguanide, is disclosed in Werner, E. A. et al, J Chem. Soc. (1922) 121:1790-1794. The compound and its preparation and use are also disclosed, for example, in U.S. Pat. No. 3,174,901.

It is postulated that metformin decreases hepatic glucose production and improves insulin sensitivity by increasing peripheral glucose uptake and utilization. Metformin can effectively inhibit hepatic glucose production and increase the sensitivity of peripheral tissues to insulin with excellent safety. Clinical studies also showed that metformin can be used in obesity, polycystic ovarian syndrome, type 1 diabetes mellitus, as well as adolescent's obesity with insulin-resistance. (Nestler, J. E., New Eng. Jour. Med. (2008) 358:47-54; Park, M. H. et al, Diabetes Care (2009) 32:1743-1745; Van Der Aa, M. et al, Nutrition & Diabetes (2016) 6, e228).

### Clinical Trial

A multicenter, international, open-label, Phase I trial is designed to evaluate the safety, tolerability, and pharmacokinetics of GDC-0077 administered orally as a single agent in patients with locally advanced or metastatic PIK3CA-mutant solid tumors, including breast cancer, and in combination with standard-of-care endocrine and targeted therapies for the treatment of locally advanced or metastatic PIK3CA-mutant hormone-receptor positive (HR+)/human epidermal growth factor receptor (EGFR) 2 negative (HER2-) breast cancer.

In one embodiment of the study, Target Population Inclusion and Exclusion Criteria are:
Determination of PIK3CA-mutant tumor status may be based on results from archival or fresh tumor tissue or ctDNA. Patients may be enrolled on the basis of local or central test results that indicate a PIK3CA mutation. PIK3CA mutations are defined as follows: H1047R/Y/L, E542K, E545K/D/G/A, Q546K/R/E/L, N345K, C420R, G1049R, R88Q, M1043I. Confirmed detection of PIK3CA mutations should be determined in a Clinical Laboratory Improvement Amendments (CLIA)-certified or equivalent laboratory.
HR+ (hormone receptor positive) is defined as expression of estrogen receptor (ER) in ≥1% of cells, or HR+ by local laboratory or regional definition.
HER2− (negative) is defined as a HER2 immunohistochemistry (IHC) score of 0 or 1+, or an IHC score of 2+ accompanied by a negative fluorescence, chromogenic, or silver in situ hybridization test indicating the absence of HER2 gene amplification, or a HER2/CEP17 ratio of <2.0, or local clinical guidelines.
If more than one test result is available for hormone receptors or HER2, and not all results meet the inclusion criterion definition, all results should be discussed with the Medical Monitor to establish eligibility of the patient.

Postmenopausal is defined as one of the following:
Age ≥60 years
Age <60 years and 12 months of amenorrhea plus follicle stimulating hormone and plasma estradiol levels within postmenopausal range by local laboratory assessment in the absence of oral contraceptive pills, hormone replacement therapy, or gonadotropin-releasing hormone (GnRH) agonist or antagonist
Prior bilateral oophorectomy

Patients must meet the following inclusion criteria for study entry:
Signed Informed Consent Form
Age ≥18 years
Evaluable or measurable disease per Response Evaluation Criteria in Solid Tumors (RECIST), Version 1.1
Eastern Cooperative Oncology Group (ECOG) performance status of 0 or 1
Life expectancy of ≥12 weeks
Adequate hematologic and organ function within 14 days prior to initiation of study treatment, defined by the following:
Absolute neutrophil count ≥1200/μL (except Arms B, E, and F, see below)
Hemoglobin ≥9 g/dL
Platelet count ≥100000/μL
Fasting glucose ≤140 mg/dL and glycosylated hemoglobin (HbAlc)<7%
Total bilirubin ≤1.5×upper limit of normal (ULN)
Serum albumin ≥2.5 g/dL
AST and ALT ≥2.5×ULN with the following exception:
Patients with documented liver metastases may have AST and/or ALT 5.0×ULN.
Serum creatinine 1.5×ULN or creatinine clearance 50 m/min on the basis of the Cockcroft-Gault glomerular filtration rate estimation:

(140−age)×(weight in kg)×(0.85 if female)

72×(serum creatinine in mg/dL)
INR <1.5×ULN and aPTT<1.5×ULN
   For patients requiring anticoagulation therapy with warfarin, a stable INR between 2-3 is required. If anticoagulation is required for a prosthetic heart valve, then stable INR between 2.5-3.5 is permitted.
Confirmation of adequate tumor tissue sample (refer to stage-specific criteria below and to the laboratory manual for instructions)
For women of childbearing potential (Stage I, Arm A and Stage II, Arms E and F only): agreement to remain abstinent (refrain from heterosexual intercourse) or use a non-hormonal contraceptive method with a failure rate of <1% per year, and agreement to refrain from donating eggs, during the treatment period and for at least 60 days after the last dose of study treatment (based on local prescribing information for fulvestrant, patients may be advised to use an effective means of contraception for up to 1 year after the last dose of fulvestrant).
   A woman is considered to be of childbearing potential if she is postmenarcheal, has not reached a postmenopausal state (12 continuous months of amenorrhea with no identified cause other than menopause), and has not undergone surgical sterilization (removal of ovaries and/or uterus).
   Examples of non-hormonal contraceptive methods with a failure rate of <1% per year include bilateral tubal ligation, male sterilization, and copper intrauterine devices.
   The reliability of sexual abstinence should be evaluated in relation to the duration of the clinical trial and the preferred and usual lifestyle of the patient. Periodic abstinence (e.g., calendar, ovulation, symptothermal, or post-ovulation methods) and withdrawal are not acceptable methods of contraception.

In one embodiment of the study, Inclusion Criteria Specific to Patients Enrolling in Stage II, Arm E are:
Female patients with histologically documented locally advanced or metastatic PIK3CA-mutant HR+/HER2− breast cancer
Pre/perimenopausal patients must be treated with GnRH or LHRH agonist therapy beginning at least 4 weeks prior Day 1 of Cycle 1 and continuing for the duration of study treatment.
Absolute neutrophil count ≥1500/μL

In one embodiment of the study, Inclusion Criteria Specific to Patients Enrolling in Stage II, Arm F are:
Female patients with histologically documented locally advanced or metastatic PIK3CA-mutant HR+/HER2− breast cancer
Pre/perimenopausal patients must be treated with GnRH or LHRH agonist therapy beginning at least 4 weeks prior Day 1 of Cycle 1 and continuing for the duration of study treatment.
Absolute neutrophil count ≥1500/μL
Patients with BMI ≥30 kg/m² or HbA1c ≥5.7% and <7% at baseline.

In one embodiment, female patients with HR+/HER2− Locally Advanced/Metastatic Breast Cancer will meet the criteria:
PIK3CA mutation in tumor tissue or ctDNA
Postmenopausal or pre/peri-menopausal on LHRH agonist
Progression during or within 12 mo of completion of adjuvant endocrine therapy
No prior systemic therapy for metastatic disease
No prior fulvestrant, SERD, PI3K, AKT, or mTOR inhibitor treatment

In one embodiment of the study, patients who meet any of the following criteria will be excluded from study entry:
Inflammatory or metaplastic breast cancer
Any history of leptomeningeal disease
Type 1 or 2 diabetes requiring anti-hyperglycemic medication
Inability or unwillingness to swallow pills
Malabsorption syndrome or other condition that would interfere with enteral absorption
Known and untreated, or active central nervous system (CNS) metastases (progressing or requiring anticonvulsants or corticosteroids for symptomatic control).
Patients with a history of treated CNS metastases are eligible, provided they meet all of the following criteria:
   Measurable or evaluable disease outside the CNS
   No ongoing requirement for corticosteroids as therapy for CNS metastases, with corticosteroids discontinued for 2 weeks prior to enrollment and no ongoing symptoms attributed to CNS metastases
   Radiographic demonstration of improvement upon the completion of CNS-directed therapy and no evidence of interim progression between the completion of CNS-directed therapy and the screening radiographic study
   The screening CNS radiographic study is 4 weeks since completion of radiotherapy
   No history of intracranial hemorrhage or spinal cord hemorrhage
Uncontrolled pleural effusion or ascites requiring recurrent drainage procedures biweekly or more frequently. Indwelling pleural or abdominal catheters may be allowed provided the patient has adequately recovered from the procedure, is hemodynamically stable and symptomatically improved, and with prior approval from the Medical Monitor
Serious infection requiring IV antibiotics within 7 days prior to Day 1 of Cycle 1
Any concurrent ocular or intraocular condition (e.g., cataract or diabetic retinopathy) that, in the opinion of the investigator and/or study ophthalmologist, would require medical or surgical intervention during the study period to prevent or treat vision loss that might result from that condition
Active inflammatory (e.g., uveitis or vitritis) or infectious (e.g., conjunctivitis, keratitis, scleritis, or endophthalmitis) conditions in either eye or history of idiopathic or autoimmune-associated uveitis in either eye
Patients requiring any daily supplemental oxygen
History of or active inflammatory disease (e.g., Crohn's disease or ulcerative colitis) or any active bowel inflammation (including diverticulitis)
   Patients currently receiving immunosuppressants (e.g., sulfasalazines) are considered to have active disease and are, therefore, ineligible.
Symptomatic hypercalcemia requiring continued use of bisphosphonate or denosumab therapy
   Bisphosphonate and denosumab therapy for bone metastases or osteopenia/osteoporosis is allowed.
Clinically significant history of liver disease, including viral or other hepatitis, current alcohol abuse, or cirrhosis
Known HIV infection
Any other diseases, active or uncontrolled pulmonary dysfunction, metabolic dysfunction, physical examination finding, or clinical laboratory finding giving reasonable suspicion of a disease or condition that contraindicates the use of an investigational drug, that may affect the interpretation of the results, or renders the patients at high risk from treatment complications
Significant traumatic injury or major surgical procedure within 4 weeks prior to initiation of GDC-0077.
Treatment with chemotherapy, immunotherapy, or biologic therapy as anti-cancer therapy within 3 weeks prior to initiation of study treatment, or endocrine therapy (e.g., tamoxifen, letrozole, anastrozole, exemestane, fulvestrant) within 2 weeks prior to initiation of study treatment, except for the following:
   Stage I, Arm A: Premenopausal patients with breast cancer may continue GnRH agonist therapy on study as long as this was initiated 4 weeks prior to Day 1 of Cycle 1.
   Kinase inhibitors, approved by regulatory authorities, may be used up to 2 weeks prior to initiation of study treatment, provided any drug-related toxicity has completely resolved and prior approval is obtained from the Medical Monitor.
   Treatment with an investigational agent within 3 weeks or five half-lives prior to initiation of study treatment, whichever is shorter.
   A shorter washout period may be allowed, if the patient has adequately recovered from any clinical relevant toxicity and with prior approval from the Medical Monitor.
Radiation therapy (other than palliative radiation to bony metastases) as cancer therapy within 4 weeks prior to initiation of study treatment
Palliative radiation to bony metastases within 2 weeks prior to initiation of GDC-0077
Unresolved toxicity from prior therapy, except for alopecia and Grade 2 peripheral neuropathy
Inability to comply with study and follow-up procedures
History of other malignancy within 5 years prior to screening, except for appropriately treated carcinoma in situ of the cervix, non-melanoma skin carcinoma, or Stage I uterine cancer
History of or active ventricular dysrhythmias or congestive heart failure requiring medication or coronary heart disease that is symptomatic,
Clinically significant electrolyte abnormalities (e.g., hypokalemia, hypomagnesemia, hypocalcemia)
Congenital long QT syndrome or QT interval corrected using Fridericia's formula (QTcF) >470 ms demonstrated by at least two ECGs >30 minutes apart, or family history of sudden unexplained death or long QT syndrome
Current treatment with medications that are well known to prolong the QT interval
Allergy or hypersensitivity to components of the GDC-0077 formulation, palbociclib (Stage I and Stage II, Arm B), letrozole (Stage I and Stage II, Arms B and C), or fulvestrant (Stage II, Arm D).
   Stage II, Arms E and F: GDC-0077 in Combination with Palbociclib and Fulvestrant:

Stage II, Arms E and F will inform the safety, tolerability, and pharmacokinetics of GDC-0077 in combination with palbociclib and fulvestrant. The combination of palbociclib and fulvestrant has been associated with a significant improvement in progression-free survival (PFS) compared with fulvestrant plus placebo in patients with HR+/HER2− metastatic breast cancer (Cristofanilli et al. 2016) and, thus, is an important standard-of-care treatment for patients.

### Stage II, Arm F: Addition of Metformin in Obese or Pre-Diabetic Patients:

Stage II, Arm F will enroll patients who are obese or pre-diabetic, defined as patients with body-mass index ≥30 kg/m² or screening HbA1c≥5.7%, who will receive metformin together with palbociclib and fulvestrant, followed by the addition of GDC-0077. Earlier administration of metformin is intended to allow sufficient time to up-titrate metformin in a tolerable manner to an effective dose and thus limit the occurrence of hyperglycemia to mild events that may be effectively managed with metformin alone, thereby limiting dose reductions or interruptions of GDC-0077. Patients with Type 1 or 2 diabetes requiring anti-hyperglycemic medication and patients with elevated fasting glucose >140 mg/dL or HbA1c ≥7% at baseline continue to be excluded from the study. In Stage II, Arm F, patients will receive metformin 500 mg total daily dose starting at Cycle 1, Day 1 and increase metformin in increments of 500 mg every 3 days (+2 days) as tolerated up to a total daily dose of 2000 mg by Cycle 1, Day 15 when GDC-0077 administration will begin. Fasting glucose levels will be assessed at baseline, and fasting glucose and insulin levels will be monitored during the study. Symptoms associated with hyperglycemia include polydipsia, polyuria, polyphagia, blurry vision, or acidosis.

Stage II, Arm E (GDC-0077 in combination with palbociclib and fulvestrant): This portion of the study will enroll patients with locally advanced or metastatic PIK3CA-mutant HR+/HER2− breast cancer. The combination of palbociclib and fulvestrant has emerged as a standard-of-care treatment option based on results from the PALOMA-3 study that demonstrated significant improvement in PFS with the addition of palbociclib to fulvestrant in patients with HR+/HER2− metastatic breast cancer that had progressed on prior endocrine therapy (Cristofanilli M et al (2016) Lancet Oncol 17:425-439).

Stage II, Arm F (GDC-0077 in combination with palbociclib, fulvestrant, and metformin): The anti-hyperglycemic medication metformin is an established standard-of-care treatment for the management of Type 2 diabetes, with an acceptable safety and tolerability profile. In addition, data from clinical trials demonstrate a benefit of metformin in diabetes prevention such that the American Diabetes Association recommends metformin be considered for diabetes prevention in at-risk patients, including those with obesity and pre-diabetes. Common metformin side effects are gastrointestinal in nature and can be minimized by use of an extended-release instead of an immediate-release formulation, low starting dose, and slow up-titration to effective dose over 1-2 weeks. Importantly, in the absence of deficient caloric intake or strenuous exercise without sufficient caloric intake, metformin does not cause hypoglycemia in patients with or without Type 2 diabetes based on its mechanism of action and lack of hyperinsulinemia (GLUCOPHAGE® U.S. Package Insert; American Diabetes Association 2015; Hostalek et al Drugs (2015) 75:1071-94).

Thus, in this portion of the study, obese or pre-diabetic patients, defined as body-mass index (BMI)≥30 kg/m² or screening HbA1c≥5.7%, will receive metformin together with palbociclib and fulvestrant followed by the addition of GDC-0077. Earlier administration of metformin is intended to allow sufficient time to up-titrate metformin in a tolerable manner to an effective dose and thus limit the occurrence of hyperglycemia while in the study to mild events that may be effectively managed with metformin alone, thereby limiting dose reductions or interruptions of GDC-0077. Patients will receive metformin at a total daily dose of 500 mg starting at Cycle 1, Day 1 and increase metformin in increments of 500 mg every 3 days (+2 days) as tolerated up to a total daily dose of 2000 mg by Cycle 1, Day 15 when GDC-0077 administration will begin.

### Study Objectives

The study will evaluate the safety, tolerability, pharmacokinetics, pharmacodynamic (PD) effects, and preliminary activity of GDC-0077 in patients with locally advanced or metastatic PIK3CA-mutant solid tumors, including breast cancer, and in combination with standard-of-care endocrine and targeted therapies for the treatment of locally advanced or metastatic PIK3CA-mutant hormone receptor-positive (HR+)/human epidermal growth factor receptor 2 negative (HER2-) breast cancer.

In one embodiment of the study, specific objectives and corresponding endpoints for the study are outlined in Table 1.

TABLE 1
Exemplary Objectives and Corresponding Endpoints

| | Corresponding Endpoints |
|---|---|
| Safety Objective | |
| (Primary Study Objective) | |
| To evaluate the safety and tolerability | Incidence and nature of DLTs |
| of GDC-0077 administered alone | Incidence, nature, and severity of adverse |
| (Stage I) or in combination with | events (AE) graded according to National |
| palbociclib and letrozole (Stages I | Cancer Institute Common Terminology Criteria |
| and II), letrozole (Stages I and II), | for Adverse Events (NCI CTCAE) v4.0 |
| fulvestrant (Stage II), palbociclib and | Change from baseline in targeted vital signs |
| fulvestrant (Stage II), or | Change from baseline in targeted clinical |
| palbociclib, fulvestrant and | laboratory test results, including |
| metformin (Stage II), including | electrocardiograms (ECGs) |
| estimation of the maximum tolerated | |
| dose (MTD; or maximum administered | |
| dose [MAD]) and characterization of | |
| dose-limiting toxicities (DLTs) to | |
| identify a recommended Phase II dose | |
| Pharmacokinetic Objectives | |
| To characterize the pharmacokinetic | Plasma concentration of GDC-0077 |
| (PK) profile of GDC-0077 | administered as a single agent (Stage I) or in |
| administered as a single agent (Stage I) | combination with palbociclib and letrozole |
| or in combination with palbociclib and | (Stages I and II), letrozole (Stages I and II), |
| letrozole (Stages I and II), letrozole | fulvestrant (Stage II), palbociclib and |
| (Stages I and II), fulvestrant (Stage II), | fulvestrant (Stage II), or palbociclib, |
| palbociclib and fulvestrant (Stage II), | fulvestrant, and metformin (Stage II) at |
| or palbociclib, fulvestrant, and | specified time points. The following PK |
| metformin (Stage II) | parameters will be determined as appropriate: |
| To characterize the PK profile of | Area under the curve (AUC) |
| palbociclib, letrozole, or fulvestrant | half-life (t_{1/2}) |
| administered in combination with | Maximum plasma concentration (Cₘₐₓ) |
| GDC-0077 | Minimum plasma concentration (Cₘᵢₙ) |
| | Additional plasma PK parameters as |
| | warranted |
| | PK parameters of palbociclib, letrozole, and |
| | fulvestrant and comparison to available |
| | historical and/or literature values |
| Exploratory Pharmacokinetic Objectives | |
| To characterize any GDC-0077 | Urine concentration of GDC-0077 at specified |
| metabolites | time points |
| To evaluate potential relationships | Determination of renal excretion fraction |
| between drug exposure and the safety | and urinary clearance |
| and activity of GDC-0077 | Plasma and urine metabolite profiling |
| To evaluate the effect of food on the | Relationship between plasma concentration or |
| pharmacokinetics of GDC-0077 | PK parameters for GDC-0077 and safety |
| To assess potential PK interactions | endpoints |
| between GDC-0077 and palbociclib, | Relationship between plasma concentration or |
| letrozole, or fulvestrant | PK parameters for GDC-0077 and activity |
| | endpoints |
| | Relationship between plasma concentrations or |
| | PK parameters for GDC-0077 during the fed |
| | and fasted states as assessed in the fulvestrant |
| | combination arm (Arm D) |
| | Plasma concentration or PK parameters for |
| | GDC-0077 administered in combination with |
| | palbociclib, letrozole, or fulvestrant compared |
| | with GDC-0077 administered as a single agent |
| Activity Objectives | |
| To make a preliminary assessment of | Objective response, defined as a complete or |
| the anti-tumor activity of GDC-0077 | partial response (PR) on two consecutive |
| administered as a single agent or in | occasions ≥4 weeks apart, as determined by |
| combination with palbociclib and | the investigator through use of RECIST v1.1 |
| letrozole, letrozole, fulvestrant, | Duration of response (DOR), defined as the |
| palbociclib and fulvestrant, or | time from the first occurrence of a documented |
| palbociclib, fulvestrant, and | objective response to disease progression, as |
| metformin in patients with locally | determined by the investigator through use of |
| advanced or metastatic PIK3CA-mutant | RECIST v1.1, or death, whichever occurs first |
| hormone-receptor positive | Clinical benefit rate (CBR) defined as the |
| (HR+)/human epidermal growth factor | percentage of patients achieving confirmed |
| receptor 2 negative (HER2−) breast | RECIST v1.1 defined complete response (CR), |
| cancer | partial response, and/or stable disease (non- |
| To evaluate changes in tumor | complete response/non-progressive disease |
| (18)F-fluorodeoxyglucose (FDG) | for patients with non-measurable disease at |
| uptake as assessed by positron emission | baseline) ≥24 weeks |
| tomography (PET) | Progression-free survival (PFS), defined as the |
| | time from the first study treatment (Day 1) to |
| | the first occurrence of disease progression, as |
| | determined by the investigator through use of |
| | RECIST v1.1, or death, whichever occurs first |
| | Change in maximum standard uptake value |
| | (SUV) of tumor regions of interest from |
| | baseline (within 14 days prior to dosing |
| | on Day 1 of Cycle 1) to approximately 2 weeks |
| | of GDC-0077 treatment |
| Exploratory Biomarker Objective | |
| To identify biomarkers that are | Correlation of efficacy with molecular markers |
| predictive of response to GDC-0077 | related to the mechanism of action of GDC- |
| (i.e., predictive biomarkers), are | 0077 that include the following: |
| associated with progression to a more | Alterations in DNA and RNA, including |
| severe disease state (i.e., prognostic | DNA mutational status, RNA expression |
| biomarkers), are associated with | levels, DNA copy number and protein |
| acquired resistance to GDC-0077, are | expression |
| associated with susceptibility to | Alteration in tumor tissue biomarkers |
| developing adverse events, can provide | including but not limited PTEN |
| evidence of GDC-0077 activity | Assessment of biomarkers (e.g., PIK3CA) in |
| (i.e., pharmacodynamic biomarkers), or | circulating tumor DNA (ctDNA) from |
| can increase the knowledge and | peripheral blood, as well as additional |
| understanding of disease biology | cancer-type specific mutations (e.g., ESRI |
| | mutations) |

### Assessment and Analysis

Clinical toxicity may not be a reliable surrogate of target modulation by GDC-0077. Therefore, PD biomarkers may be measured in tissue to determine whether clinically achievable exposures are sufficient for producing the desired effect on the intended molecular target.

Breast cancer is a heterogeneous disease, and PIK3CA mutation status has been shown to vary among patients (Cancer Genome Atlas Network 2012). In one embodiment of the study and in addition to PIK3CA mutation status, patient samples are assessed for additional biomarkers in an effort to identify factors that may correlate with the safety and efficacy of treatment with GDC-0077. Predictive biomarker samples may be collected prior to dosing to identify those patients with PIK3CA-driven pathogenesis who are most likely to respond to GDC-0077. PD biomarkers will be assessed to demonstrate evidence of biologic activity of GDC-0077 in patients, to support selection of a recommended dose and dose regimen, and to inform potential revisions to the PK sample collection schedule.

Blood samples will be collected at baseline, on study, and at disease progression. Tumor tissue will be collected at baseline and, if deemed clinically feasible, on study and/or at the time of disease progression. DNA extraction will enable analysis via next-generation sequencing (NGS) to identify germline mutations and/or somatic mutations that are predictive of response to study drug, are associated with progression to a more severe disease state, are associated with acquired resistance to study drug, are associated with susceptibility to developing adverse events, or can increase the knowledge and understanding of disease biology.

In other embodiments, biomarker and patient sample assessments may include: Tissue and Circulating Biomarker Assessments, PIK3CA Mutation Status; Pharmacodynamic Pathway Modulation; Phosphatase Tensin Homolog (PTEN) Expression Analysis; Estrogen Receptor and Progesterone Receptor (PR) Analysis; Sequencing of Genes Related to Resistance to PI3K Inhibitors; RNA and DNA Analysis; Plasma Sample for Somatic Tumor Mutation Analysis; Tumor Biopsy Sample at the Time of Disease Progression; QT/QTc Cardiotoxicity Assessments; and FDG-PET Evaluation.

Methods of Treatment with GDC-0077

The clinical trial and study design describe methods of treating patients with cancer by first administering metformin, followed by GDC-0077. Additional therapeutic agents may be part of the treatment regimen.

The invention includes a method for the treatment of cancer in a patient comprising administering a therapeutically effective amount of GDC-0077, or a pharmaceutically acceptable salt thereof, wherein the patient has been previously treated with metformin, and GDC-0077 has the structure:

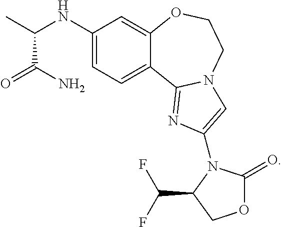 [SMILES: C[C@H](Nc1ccc2c(c1)OCCn1cc(N3C(=O)OC[C@H]3C(F)F)nc1-2)C(N)=O]

In an exemplary embodiment, GDC-0077 is administered once per day to the patient.

In an exemplary embodiment, the therapeutically effective amount of GDC-0077 is about 1 mg to about 15 mg, administered once per day.

In an exemplary embodiment, the therapeutically effective amount of GDC-0077 is about 6 mg.

In an exemplary embodiment, the therapeutically effective amount of GDC-0077 is about 9 mg.

In an exemplary embodiment, the patient has locally advanced or metastatic PIK3CA-mutant solid tumors.

In an exemplary embodiment, the patient has a cancer selected from the group consisting of breast cancer, non-small cell lung cancer, ovarian cancer, endometrial cancer, prostate cancer, and uterine cancer.

In an exemplary embodiment, the patient has breast cancer.

In an exemplary embodiment, the patient has locally advanced or metastatic PIK3CA-mutant hormone-receptor positive breast cancer.

In an exemplary embodiment, the breast cancer is HER2-negative.

In an exemplary embodiment, the patient is further administered palbociclib.

In an exemplary embodiment, the patient is further administered fulvestrant.

In an exemplary embodiment, the patient is further administered letrozole.

In an exemplary embodiment, the patient is further administered palbociclib and fulvestrant.

In an exemplary embodiment, the patient is obese or pre-diabetic.

In an exemplary embodiment, the dose or regimen of metformin is adjusted to moderate, stabilize, or diminish hyperglycemia in the patient prior to administration of GDC-0077.

In an exemplary embodiment, the blood sugar level of the patient is monitored during treatment with metformin.

In an exemplary embodiment, the patient is administered 500 mg or more of metformin daily.

In an exemplary embodiment, the patient is administered from 500 mg to 2000 mg metformin daily for about 15 days before administration of GDC-0077.

In an exemplary embodiment, the patient is administered from 500 mg to 2000 mg metformin daily beginning with the first dose administration of GDC-0077.

In an exemplary embodiment, the patient is administered from 500 mg to 2000 mg metformin daily for about 15 days before administration of palbociclib and fulvestrant, followed by administration of GDC-0077.

In an exemplary embodiment, the patient is administered metformin, palbociclib, and fulvestrant daily for about 15 days before administration of GDC-0077.

In an exemplary embodiment, the patient is further administered an additional therapeutic agent selected from the group consisting of an anti-inflammatory agent, an immunomodulatory agent, chemotherapeutic agent, an apoptosis-enhancer, a neurotropic factor, an agent for treating cardiovascular disease, an agent for treating liver disease, an anti-viral agent, an agent for treating blood disorders, an agent for treating diabetes, and an agent for treating immunodeficiency disorders.

In an exemplary embodiment, the additional therapeutic agent is selected from the group consisting of paclitaxel, anastrozole, exemestane, cyclophosphamide, epirubicin, fulvestrant, letrozole, palbociclib, gemcitabine, trastuzumab (HERCEPTIN®, Genentech), trastuzumab emtansine (KADCYLA®, Genentech), pegfilgrastim, filgrastim, lapatinib, tamoxifen, docetaxel, toremifene, vinorelbine, capecitabine, and ixabepilone.

In an exemplary embodiment, the additional therapeutic agent is a selective estrogen receptor modulator (SERM) or a selective estrogen-receptor degrader (SERD).

In an exemplary embodiment, the additional therapeutic agent is a CDK 4/6 inhibitor.

In an exemplary embodiment, the CDK 4/6 inhibitor is selected from palbociclib, ribociclib, and abemaciclib (LY283519, VERZENIO®, Eli Lilly).

In an exemplary embodiment, the additional therapeutic agent is selected from the group consisting of a phosphoinositide 3-kinase (PI3K)/mTOR pathway inhibitor selected from everolimus, temsirolimus, BEZ235 (dactolisib), BYL719 (alpelisib), GDC0032 (taselisib), BKM120 (buparlisib), BGT226, GDC0068 (ipatasertib), GDC-0980 (apitolisib), GDC0941 (pictilisib), INK128 (MLN0128), INK1117, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, Cal10 (idelalisib), PWT33597, CU-906, AZD-2014 and CUDC-907.

The invention includes all reasonable combinations, and permutations of the features, of the exemplary embodiments of the methods described herein.

### EXAMPLES

### Example 1 Formulation, Packaging, and Handling

GDC-0077 (CAS Registry Number 2060571-02-8) Drug Product is provided as a tablet in two tablet strengths: 1 mg and 5 mg. The 1-mg tablet is a white to off-white, plain or speckled, triangle or round-shaped tablet, and the 5-mg tablet is a white to pink, plain or speckled, round-shaped tablet. The excipients in GDC-0077 Drug Product include microcrystalline cellulose, lactose, magnesium stearate, and sodium starch glycolate.

The starting dose of GDC-0077 to be evaluated in the single-agent, dose-escalation portion of this study is about 6 to 9 mg administered daily by mouth (PO). Patients may be instructed as to the number and strength of tablets to take, according to their assigned dose level and schedule. In one embodiment of the study, GDC-0077 is taken on an empty stomach (i.e., approximately 1 hour before or 2 hours after a meal) and at approximately the same time each day ±2 hours.

GDC-0077 may be administered as a single agent (Stage I, Arm A) and in combination with the following standard-of-care therapies for HR+ breast cancer: palbociclib and letrozole (Stages I and II, Arm B), letrozole (Stages I and II, Arm C), fulvestrant (Stage II, Arm D), and palbociclib and fulvestrant (Stage II, Arm E and Arm F). In addition, patients enrolled in Stage II, Arm F will also receive metformin as part of the study treatment.

Palbociclib may be used as 75-mg, 100-mg, and 125-mg capsules. In one embodiment of the study, palbociclib is administered at its label-recommended starting dose of 125 mg PO daily on Days 1-21 of each 28-day cycle. Patients will be instructed to take palbociclib with food and at approximately the same time each day ±2 hours, unless otherwise instructed.

Letrozole is available as 2.5-mg tablets in a bottle or blister pack. In one embodiment of the study, letrozole is administered at 2.5 mg PO daily. Unless otherwise instructed, patients will take letrozole doses on an empty stomach (i.e., 1 hour before or 2 hours after a meal) and at approximately the same time each day ±2 hours.

Fulvestrant is available as sterile single-patient prefilled syringes containing 50 mg/mL fulvestrant as a 5-mL injection in a carton. In one embodiment of the study, fulvestrant 500 mg is administered intramuscularly in the buttocks in the clinic on Days 1 and 15 of Cycle 1. For subsequent cycles, patients will receive fulvestrant in the clinic on Day 1 of each cycle or approximately every 4 weeks.

Metformin (FORTAMET®, GLUCOPHAGE®, GLUCOPHAGE XR®, GLUMETZA®, RIOMET®) may be supplied as 500-mg extended-release tablets in a bottle, or supplied by the study sites. In one embodiment of the study, metformin is administered at a total daily dose of 500 mg PO starting at Cycle 1, Day 1 and increased by 500 mg every 3 days (+2 days) as tolerated up to a total daily dose of 2000 mg PO by Cycle 1, Day 15.

### Example 2 Dosage, Administration, and Compliance

In Stage I, Arm A, the starting dose of GDC-0077 is 6 mg PO QD. On Day 1 of Cycle 1, a single dose of GDC-0077 will be administered to patients in a clinical setting that can accommodate frequent blood draws over a period of up to 48 hours after the morning dose is administered. QD dosing of GDC-0077 will begin on Day 8 of Cycle 1. The length of Cycle 1 will be 35 days, and all subsequent cycles (Cycles 2) will be 28 days in length.

In Stage I, Arm A backfill cohorts, Arms B and C, and Stage II, Arms B, C, and D, QD dosing of GDC-0077 will begin on Day 1 of Cycle 1 and each cycle (Cycles 1) will be 28 days in length.

Patients will take GDC-0077 at the same time of day ±2 hours, unless otherwise instructed. Patients will be instructed as to the number and strength of tablets to take, according to their assigned dose level and schedule. Patients will be asked to record the time and date that they take each dose in a medication diary.

Unless otherwise instructed, GDC-0077 should be taken on an empty stomach (i.e., approximately 1 hour before or 2 hours after a meal), except on days of extensive PK sampling (Days 1 and 15 of Cycle 1) when administration will be under fasted conditions. For administration under fasted conditions, patients will fast overnight for at least 8 hours before dosing and 3 hours post-dose and will refrain from drinking water from 1 hour before and until 1 hour after dosing, with the exception of GDC-0077 administration when the tablets will be swallowed whole (not chewed) with 240 mL (8 fluid ounces) of water.

PK samples will be collected at the same time as other blood tests are performed, including fasting lipid panels. Patients will be instructed to hold the morning dose of GDC-0077 until after PK blood samples have been obtained.

For patients enrolled in dose-escalation cohorts, Cycle 1 of Stage I, Arm A will be 35 days in length and will begin with a PK evaluation, during which all patients will receive a single fasting dose of GDC-0077 on Day 1 at their assigned dose level. The initial dose will be followed by a 7-day washout and frequent PK sampling up to 48 hours to determine the single-dose PK properties of GDC-0077 in humans. Urine samples will be collected up to 8 hours after the first dose to determine urinary elimination of GDC-0077. In Cycle 1, continuous GDC-0077 QD dosing will begin on Day 8 and will continue for 4 weeks (Days 8-35). Subsequent cycles (Cycles 2) will be 28 days in length (4 weeks of QD dosing with GDC-0077). For patients enrolled in backfill cohorts, daily dosing of GDC-0077 will begin on Day 1 of Cycle1, and all cycles will be 28 days in length.

Beginning with Cycle 1 of Stages I and II, Arm B (GDC-0077 Dose-Escalation and Dose-Cohort Expansion in Combination with Palbociclib and Letrozole), all cycles will be 28 days in length. Patients will receive GDC-0077 at their assigned dose level on Days 1-28 along with palbociclib PO QD on Days 1-21, and letrozole PO QD on Days 1-28 of each 28-day cycle. Patients will take GDC-0077, letrozole, and palbociclib with food per the local prescribing information for palbociclib. On study visit days, GDC-0077, palbociclib, and letrozole will be administered in the clinic, and patients should be instructed to fast (overnight for 8 hours) prior to the pre-dose blood draw. Local laboratory results, including CBC, chemistry panel, and glucose, may be reviewed prior to dosing.

In the event palbociclib administration is held due to an adverse event in a given cycle, the next dosing cycle should not begin until administration of palbociclib can be resumed. As such, the current cycle may be extended past 28 days, and the patient may continue to receive GDC-0077 and letrozole. Day 1 of the next cycle should correspond to the time point at which administration of palbociclib is resumed. At that time, palbociclib may be administered with GDC-0077 and letrozole.

Beginning with Cycle 1 of Stages I and II, Arm C (GDC-0077 Dose-Escalation and Dose-Cohort Expansion in Combination with Letrozole), all cycles will be 28 days in length. Patients will receive GDC-0077 at their assigned dose level on Days 1-28 along with letrozole 2.5 mg PO QD on Days 1-28 of each 28-day cycle. Patients will take the GDC-0077 and letrozole doses on an empty stomach (i.e., 1 hour before or 2 hours after a meal), except on Day 1 of Cycles 1 and 2 when patients will receive the doses under fasted conditions. On study visit days, GDC-0077 and letrozole will be administered in the clinic.

Beginning in Cycle 1 of Stage II, Arm D (GDC-0077 Dose Cohort Expansion in Combination with Fulvestrant), patients will receive GDC-0077 at or below the MTD or MAD determined in Stage I, Arm C. Once the GDC-0077 dose in combination with fulvestrant is deemed tolerable over the first cycle of treatment in 6 patients (safety run-in), additional patients will be enrolled. During Cycle 1, patients will be assigned in an alternating fashion to either Day 1 (odd-numbered patients) or Day 8 (even-numbered patients) for the food-effect assessment. On Day 1 (odd-numbered patients) or Day 8 (even-numbered patients), GDC-0077 will be administered under fed conditions. For dosing under fed conditions, patients will fast overnight for 8 hours before the standard high-fat meal provided at the study site (see laboratory manual). Patients should start a standard high-fat meal 30 minutes prior to administration of GDC-0077. Patients should consume the whole meal in 30 minutes. GDC-0077 should be administered 30 minutes after start of the meal with 240 mL (8 ounces) water. No food should be allowed until >3 hours post-dose. Water is not allowed for 1 hour before and 1 hour after drug administration, with the exception of 240 mL (8 fluid ounces) of water intake required for administration of GDC-0077. On Day 1 (even-numbered patients) or Day 8 (odd-numbered patients) and Day 15, GDC-0077 will be administered under fasting conditions. Patients will fast overnight for at least 8 hours before dosing and 3 hours post-dose; patients will refrain from drinking water from 1 hour before and until 1 hour after dosing, with the exception of GDC-0077 administration when the tablet will be swallowed whole with 240 mL (8 fluid ounces) of water. On the day of GDC-0077 dosing in the clinic, patients will receive a standard low-fat meal at 3 hours post-dose. Unless otherwise instructed, all other doses will be taken on an empty stomach (approximately 1 hour before or 2 hours after a meal).

Patients will receive fulvestrant 500 mg, administered intramuscularly in the buttocks slowly (1-2 minutes per injection) as two 5-mL injections (one in each buttock), in the clinic on Days 1 and 15 of Cycle 1. For subsequent cycles (Cycles 2), patients will receive fulvestrant via intramuscular injections as described above in the clinic on Day 1 of each cycle. Patients who received fulvestrant within 4 weeks of initiating study treatment will receive fulvestrant 500 mg on Day 1 of each Cycle starting in Cycle 1.

Beginning with Cycle 1 of Stage II, Arm E (GDC-0077 Dose-Cohort Expansion in Combination with Palbociclib and Fulvestrant), cycles will be approximately 28 days in length. Patients will receive GDC-0077 at their assigned dose level on Days 1-28 along with palbociclib PO (orally) QD (daily) on Days 1-21, and fulvestrant via intramuscular injection in the clinic on Days 1 and 15 of Cycle 1. For subsequent cycles (Cycles 2), patients will receive fulvestrant via intramuscular injections in the clinic approximately every 4 weeks. Patients who received fulvestrant within 4 weeks of initiating study treatment will receive fulvestrant on Day 1 of Cycle 1, and approximately every 4 weeks thereafter.

Patients will take GDC-0077 and palbociclib with food per the local prescribing information for palbociclib. On study visit days, GDC-0077 and palbociclib will be administered in the clinic and patients should be instructed to fast (overnight for 8 hours) prior to the pre-dose blood draw. In the event palbociclib administration is held due to an adverse event in a given cycle, the next dosing cycle should not begin until administration of palbociclib can be resumed. As such, the current cycle may be extended past 28 days, and the patient may continue to receive GDC-0077. Day 1 of the next cycle should correspond to the time point at which administration of palbociclib is resumed. At that time, palbociclib may be administered with GDC-0077. Fulvestrant will continue to be administered approximately every 4 weeks, independently from the start of the cycle.

Beginning with Cycle 1 of Stage II, Arm F (GDC-0077 Dose-Cohort Expansion in Combination with palbociclib, fulvestrant, and metformin), cycles will be approximately 28 days in length. Patients will receive palbociclib PO QD on Days 1-21 beginning in Cycle 1 and fulvestrant via intramuscular injection in the clinic on Days 1 and 15 of Cycle 1. For subsequent cycles (Cycles 2), patients will receive fulvestrant via intramuscular injections in the clinic approximately every 4 weeks. Patients who received fulvestrant within 4 weeks of initiating study treatment will receive fulvestrant on Day 1 of Cycle 1 and approximately every 4 weeks thereafter. In addition, patients will also receive metformin at a total daily dose of 500 mg starting at Cycle 1, Day 1, with an increase by 500 mg approximately every 3 days (+2 days) as tolerated up to a total daily dose of 2000 mg by Cycle 1, Day 15. Patients will receive GDC-0077 at their assigned dose level starting on Day 15 of Cycle 1. For subsequent cycles (Cycle ≥2), patients will receive GDC-0077 on Days 1-28.

Patients will take GDC-0077, palbociclib, and metformin with food per the local prescribing information for palbociclib and metformin. On study visit days, GDC-0077 and palbociclib will be administered in the clinic and patients should be instructed to fast (overnight for 8 hours) prior to the pre-dose blood draw. In the event palbociclib administration is held due to an adverse event in a given cycle, the next dosing cycle should not begin until administration of palbociclib can be resumed. As such, the current cycle may be extended past 28 days, and the patient may continue to receive GDC-0077 and metformin. Day 1 of the next cycle should correspond to the time point at which administration of palbociclib is resumed. At that time, palbociclib may be administered with GDC-0077 and metformin. Fulvestrant will continue to be administered approximately every 4 weeks, independently from the start of the cycle.

### Example 3 Study Design

This clinical trial is an open-label, multicenter, Phase I study designed to evaluate the safety, tolerability, and pharmacokinetics of GDC-0077 administered orally as a single agent in patients with locally advanced or metastatic PIK3CA-mutant solid tumors, including breast cancer, and in combination with standard-of-care endocrine and targeted therapies for the treatment of locally advanced or metastatic PIK3CA-mutant hormone-receptor positive (HR+)/human epidermal growth factor receptor 2 negative (HER2-) breast cancer.

Patients will be enrolled in two stages: a dose-escalation stage (Stage I) and an expansion stage (Stage II). Patients will be assigned to one of six regimens: GDC-0077 as a single agent (Arm A), GDC-0077 in combination with palbociclib and letrozole (Arm B), GDC-0077 in combination with letrozole (Arm C), GDC-0077 in combination with fulvestrant (Arm D), GDC-0077 in combination with palbociclib and fulvestrant (Arm E), and GDC-0077 in combination with palbociclib, fulvestrant, and metformin (Arm F). Cycle 1 in the dose-escalation cohorts of Arm A will be 35 days in length; all other cycles will be 28 days in length.

Stage I uses a 3+3 dose-escalation design to assess the safety, tolerability, and pharmacokinetics of GDC-0077 administered as a single agent in locally advanced or metastatic PIK3CA-mutant solid tumors, including breast cancer. The starting dose of GDC-0077 in the single-agent dose escalation will be 6 mg. After the dose-limiting toxicity (DLT) evaluation of at least two dose levels of single-agent GDC-0077 has been completed in Arm A and all relevant single-agent safety and pharmacokinetic (PK) data have been thoroughly reviewed with the investigators, the safety, tolerability, and pharmacokinetics of GDC-0077 administered in combination with the standard-of-care regimen of palbociclib and letrozole (Arm B) or letrozole alone (Arm C) will be evaluated using the same 3+3 dose-escalation design in locally advanced or metastatic PIK3CA-mutant HR+/HER2− breast cancer. The starting dose of GDC-0077 in combination with palbociclib and letrozole (Arm B) will be 3 mg, one dose level lower than the starting dose in the GDC-0077 single agent dose escalation (Arm A). The starting dose of GDC-0077 in combination with letrozole (Arm C) will not exceed the starting dose of 6 mg in the GDC-0077 single-agent dose escalation (Arm A) and, based on available PK and safety data, may be lower than the starting dose for Arm A. During the dose-escalation stage, cohorts of 3-6 patients each will be evaluated at escalating dose levels of GDC-0077 to determine the maximum tolerated dose (MTD) or maximum administered dose (MAD) for GDC-0077 as a single agent and in combination with palbociclib and letrozole, or letrozole.

To acquire additional PK and safety data, and tumor pharmacodynamic (PD) data related to the mechanism of action of GDC-0077, patients with locally advanced or metastatic PIK3CA-mutant breast cancer (Arm A) or PIK3CA-mutant HR+/HER2− breast cancer (Arm C) may be enrolled to backfill cohorts (Stage I, Arm A or C) at dose levels that have been shown not to exceed MTD based on the dose-escalation criteria described below. Tumor biopsies prior to starting treatment and after approximately 2 weeks of once daily (QD) study treatment administration are required for patients enrolled to backfill cohorts. The Sponsor's decision to open backfill cohorts at specific dose levels will be based on available PK and safety data. Backfill cohorts may enroll up to approximately 3-6 patients per dose level to be evaluated and may not be opened at all dose levels evaluated in dose escalation. Additional patients may be enrolled to replace patients whose pre-treatment or on-treatment biopsies have insufficient tumor tissue. For the purposes of dose-escalation decisions, patients enrolled in backfill cohorts will not be included as part of the DLT-evaluable population.

Once the MTD or MAD for GDC-0077 in combination with palbociclib and letrozole has been established (Stage I, Arm B), approximately 20 additional patients may be enrolled in a dose-cohort expansion (Stage II, Arm B) to further assess the safety, tolerability, pharmacokinetics, and preliminary anti-tumor activity of GDC-0077 administered at or below the MTD or MAD in combination with palbociclib and letrozole from Stage I in locally advanced or metastatic PIK3CA-mutant HR+/HER2− breast cancer.

Once the MTD or MAD for GDC-0077 in combination with letrozole has been established (Stage I, Arm C), additional patients may be enrolled in a dose-cohort expansion (Stage II, Arm C) to further assess the safety, tolerability, pharmacokinetics, and preliminary anti-tumor activity of GDC-0077 administered at or below the MTD or MAD in combination with letrozole from Stage I in locally advanced or metastatic PIK3CA-mutant HR+/HER2− breast cancer.

Once the MTD or MAD for GDC-0077 has been established in Stage I, Arm C, patients may be enrolled in a dose-cohort expansion (Stage II, Arm D) to assess the safety, tolerability, pharmacokinetics, and preliminary anti-tumor activity of GDC-0077 administered at or below the MTD or MAD determined in Stage I, Arm C in combination with fulvestrant in locally advanced or metastatic PIK3CA-mutant HR+/HER2− breast cancer. In Stage II, Arm D, the first 6 patients enrolled (safety run-in) will be evaluated for safety and tolerability during the first Cycle of treatment (Days 1-28) prior to enrolling additional patients.

In addition, once the MTD or MAD for GDC-0077 has been established in Stage I, Arm B, approximately 20 patients each may be enrolled in dose-cohort expansions (Stage II, Arm E and Arm F) to assess the safety, tolerability, pharmacokinetics, and preliminary anti-tumor activity of GDC-0077 (administered at or below the MTD or MAD determined in Stage I, Arm B) in combination with palbociclib and fulvestrant in locally advanced or metastatic PIK3CA-mutant HR+/HER2− breast cancer. Arm F will enroll obese and pre-diabetic patients, who will receive the anti-hyperglycemic medication metformin starting at Cycle 1, Day 1 and GDC-0077 starting at Cycle 1, Day 15. Obese and pre-diabetic patients will be defined as those patients with body mass index (BMI) 30 kg/m² or screening HbA1c 5.7% at baseline. In Stage II, Arm E and Arm F, the first 3 patients in each cohort (safety run-in) for a total of 6 patients will be evaluated for safety and tolerability during the first Cycle of treatment (Days 1-28) prior to enrolling additional patients in either arm.

The study consists of a screening period of up to 28 days, a treatment period, and a safety follow-up period of 30 days, or until initiation of another anti-cancer therapy, whichever occurs first. All patients will be closely monitored for adverse events throughout the study and for at least 30 days after the last dose of study treatment, or until initiation of another anti-cancer therapy, whichever occurs first. Adverse events will be graded according to the National Cancer Institute Common Terminology Criteria for Adverse Events (NCI CTCAE), Version 4.0.

To characterize the PK properties of GDC-0077, blood samples will be taken at various time points before and after dosing.

In the absence of unacceptable toxicities and unequivocal disease progression as determined by the investigator, patients may continue treatment with GDC-0077 until the end of the study.

Dose-Escalation Stage: Patients will be enrolled in the dose-escalation stage (Stage I) across three arms. Cohorts of at least 3 patients each will be treated at escalating doses of GDC-0077 as a single agent or as part of a combination regimen in accordance with the dose-escalation rules described below. Enrollment of the first 2 patients in all dose-escalation cohorts will be separated by at least 24 hours. Patients will be closely monitored for adverse events during a DLT assessment window. The DLT assessment window for Stage I, Arm A (GDC-0077 single agent) is defined as Days 1-35 of Cycle 1. The DLT assessment window for Stage I, Arm B (GDC-0077 in combination with palbociclib and letrozole) or Arm C (GDC-0077 in combination with letrozole) is defined as Days 1-28 of Cycle 1. Adverse events identified as DLTs, as defined below, will be reported to the Sponsor within 24 hours.

Patients who discontinue from the study prior to completing the DLT assessment window for reasons other than a DLT will be considered non-evaluable for dose-escalation decisions and MTD or MAD assessments, and will be replaced by an additional patient at that same dose level. In Stage I, Arm A, patients who miss more than 3 doses of GDC-0077 during the DLT assessment window for reasons other than a DLT will also be replaced. Patients who miss more than 3 doses of GDC-0077 or letrozole (Stage I, Arm B or C), or more than 7 doses of palbociclib (Stage I, Arm B) during the DLT assessment window for reasons other than a DLT will also be replaced. Patients who receive supportive care during the DLT assessment window that confounds the evaluation of DLTs (not including supportive care described below as part of the DLT definition) may be replaced at the discretion of the Medical Monitor. To define the DLT for GDC-0077 in combination with palbociclib and letrozole, patients should not be prophylactically prescribed growth factor support during the DLT assessment window.

Expansion Stage: A number of patients will be enrolled in the expansion stage (Stage II). In Stage II, Arm B, patients with locally advanced or metastatic PIK3CA-mutant HR+/HER2− breast cancer will be treated at or below the GDC-0077 MTD or MAD in combination with palbociclib and letrozole determined in Stage I, Arm B to obtain additional safety, tolerability, and PK data, and preliminary evidence of clinical activity. In Stage II, Arm C, patients with locally advanced or metastatic PIK3CA-mutant HR+/HER2− breast cancer will be treated at or below the GDC-0077 MTD or MAD determined in Stage I, Arm C in combination with letrozole to obtain additional safety, tolerability, and PK data, and preliminary evidence of clinical activity.

In Stage II, Arm D, patients with locally advanced or metastatic PIK3CA-mutant HR+/HER2− breast cancer will be treated at or below the GDC-0077 MTD or MAD determined in Stage I, Arm C in combination with fulvestrant to obtain additional safety, tolerability, and PK data, and preliminary evidence of clinical activity. In Stage II, Arm D, the first 6 patients enrolled (safety run-in) will be evaluated for safety and tolerability during the first Cycle of treatment (Days 1-28) prior to enrolling additional patients.

In Stage II, Arm E, patients with locally advanced or metastatic PIK3CA-mutant HR+/HER2− breast cancer will be treated with GDC-0077 (at or below the GDC-0077 MTD or MAD determined in Stage I, Arm B) in combination with palbociclib and fulvestrant to obtain additional safety, tolerability, and PK data, and preliminary evidence of clinical activity. The first 3 patients enrolled (safety run-in) for a total of 6 patients between Arms E and F will be evaluated for safety and tolerability during the first cycle of treatment (Days 1-28) prior to enrolling additional patients.

In Stage II, Arm F, obese or pre-diabetic patients with locally advanced or metastatic PIK3CA-mutant HR+/HER2− breast cancer will be treated with GDC-0077 (at or below the GDC-0077 MTD or MAD determined in Stage I, Arm B) in combination with palbociclib, fulvestrant, and metformin to obtain additional safety, tolerability, and PK data, and preliminary evidence of clinical activity. Obese and pre-diabetic patients will be defined as those patients with BMI >30 kg/m² or screening HbA1c >5.7% at baseline. Palbociclib, fulvestrant, and metformin will start at Cycle 1, Day 1 and GDC-0077 will start at Cycle 1, Day 15. The first 3 patients enrolled (safety run-in) for a total of 6 patients between Arms E and F will be evaluated for safety and tolerability during the first Cycle of treatment (Days 1-28) prior to enrolling additional patients.

If the frequency of Grade 3 or 4 toxicities or other unacceptable toxicities at the initial expansion-stage dose level suggests that the safety or tolerability of the selected GDC-0077 dose in the combination regimen is unacceptable, accrual at that dose level will be halted and patients who continue on study treatment will be allowed to reduce the GDC-0077 dose. Consideration will then be given to enrolling patients in an expansion cohort at a lower dose level.

### Example 4 Statistical Methods

Primary Analysis: Safety may be assessed through summaries of adverse events, changes in laboratory test results, and changes in vital signs. All patients who receive any amount of study treatment will be included in the safety analyses.

GDC-0077 exposure, including the proportion of patients with dose modifications, will be summarized by assigned dose level and cohort.

All collected adverse event data will be listed by study site, patient number, and cycle. All adverse events occurring on or after treatment on Day 1 will be summarized by mapped term, appropriate thesaurus levels, and NCI CTCAE v4.0 toxicity grade. In addition, all serious adverse events, including deaths, will be listed separately and summarized.

QT/QTc data will be analyzed using the E14 guidelines and may include analyses of central tendency, categorical analyses, analysis of the relationship between drug exposure and QT/QTc interval changes, and morphologic analyses of ECG waveforms.

Determination of Sample Size: The final analysis will be based on patient data collected through patient discontinuation or study discontinuation, whichever occurs first. In general, data will be summarized as warranted, and listings will be used in place of tables when the samples sizes are small. Continuous variables will be summarized using means, standard deviations, median, and ranges; categorical variables will be summarized using counts and percentages.

This study is intended to obtain preliminary safety, PK, PD, and activity information in the safety-evaluable population. The sample sizes do not reflect any explicit power and type I error considerations

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for the treatment of cancer in a patient comprising administering a therapeutically effective amount of GDC-0077, or a pharmaceutically acceptable salt thereof, wherein the patient has been previously treated with metformin, and GDC-0077 has the structure:

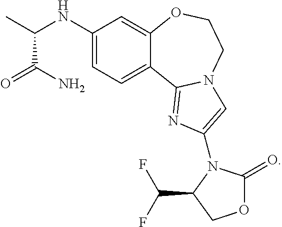 [SMILES: C[C@H](Nc1ccc2c(c1)OCCn1cc(N3C(=O)OC[C@H]3C(F)F)nc1-2)C(N)=O]

2. The method of claim 1 wherein GDC-0077 is administered once per day to the patient.

3. The method of claim 1 wherein the therapeutically effective amount of GDC-0077 is about 1 mg to about 15 mg, administered once per day.

4. (canceled)

5. The method of claim 3 wherein the therapeutically effective amount of GDC-0077 is about 9 mg.

6. The method of claim 1, wherein the patient has locally advanced or metastatic PIK3CA-mutant solid tumors.

7. The method of claim 1, wherein the patient has a cancer selected from the group consisting of breast cancer, non-small cell lung cancer, ovarian cancer, endometrial cancer, prostate cancer, and uterine cancer.

8. The method of claim 7 wherein the patient has breast cancer.

9. The method of claim 8 wherein the patient has locally advanced or metastatic PIK3CA-mutant hormone-receptor positive breast cancer.

10. The method of claim 8 wherein the breast cancer is HER2-negative.

11.-13. (canceled)

14. The method of claim 1, wherein the patient is further administered palbociclib and fulvestrant.

15. The method of claim 1, wherein the patient is obese or pre-diabetic.

16. The method of claim 1, wherein the dose or regimen of metformin is adjusted to moderate, stabilize, or diminish hyperglycemia in the patient prior to administration of GDC-0077.

17. The method of claim 1, wherein the blood sugar level of the patient is monitored during treatment with metformin.

18. The method of claim 1, wherein the patient is administered 500 mg or more of metformin daily.

19. The method of claim 1, wherein the patient is administered from 500 mg to 2000 mg metformin daily for about 15 days before administration of GDC-0077.

20. The method of claim 1, wherein the patient is administered from 500 mg to 2000 mg metformin daily beginning with the first dose administration of GDC-0077.

21. The method of claim 1, wherein the patient is administered from 500 mg to 2000 mg metformin daily for about 15 days before administration of palbociclib and fulvestrant, followed by administration of GDC-0077.

22. The method of claim 1, wherein the patient is administered metformin, palbociclib, and fulvestrant daily for about 15 days before administration of GDC-0077.

23. The method of claim 1 wherein the patient is further administered an additional therapeutic agent selected from the group consisting of an anti-inflammatory agent, an immunomodulatory agent, chemotherapeutic agent, an apoptosis-enhancer, a neurotropic factor, an agent for treating cardiovascular disease, an agent for treating liver disease, an anti-viral agent, an agent for treating blood disorders, an agent for treating diabetes, and an agent for treating immunodeficiency disorders.

24. The method of claim 23 wherein the additional therapeutic agent is selected from the group consisting of paclitaxel, anastrozole, exemestane, cyclophosphamide, epirubicin, fulvestrant, letrozole, palbociclib, gemcitabine, trastuzumab, trastuzumab emtansine, pegfilgrastim, filgrastim, lapatinib, tamoxifen, docetaxel, toremifene, vinorelbine, capecitabine, and ixabepilone.

25.-30. (canceled)
